(19) United States
(12) Patent Application Publication
(10) Pub. No.: US 2024/0316309 A1
(43) Pub. Date: Sep. 26, 2024

(54) THERAPEUTIC TOUCHING DEVICE

(71) Applicant: ATATÜRK ÜNIVERSITESI BILIMSEL ARASTIRMA PROJELERI BIRIMI, Yakutiye, Erzurum (TR)

(72) Inventors: Zeynep KARAMAN ÖZLÜ, Yakutiye, Erzurum (TR); Ibrahim ÖZLÜ, Yakutiye, Erzurum (TR); Gülistan UYMAZ ARAS, Yakutiye, Erzurum (TR)

(21) Appl. No.: 18/574,780

(22) PCT Filed: Apr. 27, 2022
(86) PCT No.: PCT/TR2022/050385

§ 371 Date: Dec. 28, 2023

(30) Foreign Application Priority Data
Dec. 10, 2021 (TR) 2021/019643

(51) Int. Cl. A61M 21/02; A61M 21/00

(52) U.S. Cl. CPC A61M 21/02; A61M 2021/0022; A61M 2205/103; A61M 2205/18; A61M 2205/33; A61M 2205/502; A61M 2210/08; A61M 2230/06; A61M 2230/20; A61M 2230/50

(57) ABSTRACT

A therapeutic touching device that increases the moral support, makes a person more positive, accelerates the healing process and makes individuals feel happier, and shows vital signs. The device includes a pulse meter that is on the touching device and positioned to touch the patient's hand, a thermometer that allows the measurement of the patient's temperature, and an oximeter that allows the measurement of the oxygen level in the patient's blood. A microprocessor provides data collection and evaluation via the pulse meter, thermometer and oximeter, checks whether the pulse, temperature and oxygen data can be collected, provides a warning if the pulse, temperature and oxygen data are below or above a certain value, and enables the touching device to be moved by motors. A display panel controlled by a microprocessor provides the display of pulse, temperature and oxygen data and provides warnings to the user.

### FIELD OF THE INVENTION

The present invention relates to a therapeutic touching device that gives the warmth and support of a person, also shows his/her vital signs and can be used in the field of health, in the intensive care unit, in need of palliative care, in home care support, in elderly individuals living alone, in children and adults living alone, and in all individuals who need moral support with its abovementioned properties.

### STATE OF THE ART

Individuals sometimes move away from being healthy and face a disease that requires care and treatment throughout their lives. Care and treatment of critically ill patients are provided in intensive care units. Entry to the intensive care unit is a stressful experience for both patients and their families (Turan, 2015). In this stressful process, the inability of individuals who are not with their families to receive moral support brings some complications that come with stress (Amaya-Montaya et al., 2020). There are studies that touch has positive effects on life energy and positive effects on wound healing and pain treatment (Midilli et al. 2019; O'Mathúna, 2016; Sa\check{g}kal et al. 2013). However, it is reported that therapeutic touch reduces anxiety and stress in patients who will undergo surgery. (Yilmaz et al. 2016; Brand and et al., 2013; Ramnarine-Singh, 1999). It is thought from this aspect that our invention will be beneficial in patients who receive mild sedation in the operating room, patients who will undergo spinal surgery, and patients who will be operated on with an open consciousness. Furthermore, studies have reported that elderly individuals become lonely and more fragile over time, thus experiencing depression and worsening the existing comorbidity. It was determined that therapeutic touch reduced the frailty and the level of anxiety and depression experienced in elderly individuals (Özdil et al. 2020). Some images have emerged during the pandemic process; showing how important the feeling of spirituality and touch is in the treatment of patients hospitalized in the intensive care unit.

### REFERENCES

Amaya-Montoya M, Pérez-Londoño A, Guatibonza-García V, Vargas-Villanueva A, Mendivil CO. Cellular Senescence as a Therapeutic Target for Age-Related Diseases: A Review. Adv Ther. 2020 April; 37(4):1407-1424.
Brand L R, Munroe D J, Gavin J. The effects of hand massage on preoperative anxiety in ambulatory surgery patients. AORN J 2013; 97:708-717.
Midilli T S, Eşer I, Yücel Ş. Cerrahi Kliniklerinde Çlisan Hemşirelerin A\check{g}ri Yönetiminde Nonfarmakolojik Yöntemleri Kullanma Durumlari ve Etkileyen Faktörler. Acibadem Üniversitesi Sa\check{g}lik Bilimleri Dergisi. 2019; 1:60-66.
O'Mathúna D P. WITHDRAWN: Therapeutic touch for healing acute wounds. Cochrane Database Syst Rev. 2016 Sep. 1; 9(9):CD002766.
Özdil K, Muz G, Öztürk GK. Yaşlilarda Farmakolojik Olmayan Uygulamalar ve Etkisinin Incelenmesine Yönelik Türkiye'deki Mevcut Tezlerin De\check{g}erlendirilmesi. HUHEMFAD-JOHUFON 2020,7(3),196-205.
Ramnarine-Singh S. The surgical significance of therapeutic touch. AORN J 1999; 69:358-369.
Sa\check{g}kal T, Eşer I, Uyar M. Reiki Dokunma Terapisinin A\check{g}ri ve Anksiyete Üzerine Etkisi. Spatula D D. 2013; 3(4):141-146.
Turan, N. Yo\check{g}un Bakim Ünitesinde Terapötik Dokunmanin Önemi. Acibadem Üniversitesi Sa\check{g}lik Bilimleri Dergisi. 2015; 6(3):134-9.
Yilmaz E, Birer Z, Baydur H. Katarakt Ameliyati Esnasinda Uygulanan Terapotik Dokunmanin Kaygi ve Hasta Memnuniyetine Etkisi. Journal of Clinical and Experimental Investigations. 2016; 7(1): 52-62.

As a result of the patent researches, no therapeutic robotic hand application was found. Robotic hands in the field are designed for the application of prosthesis or for performing activities that facilitate daily life or for massaging the hands of the person. The designs that exist as such are not qualified to give the therapeutic human warmth and touch that can be used by individuals living alone or being followed in the intensive care unit.

Abstract of the application numbered CN204080545U, which emerged as a result of technical research is as follows; “The invention relates to a kind of therapeutic massage wallpaper. The utility model wallpaper has a multi-layer composite structure, it has top layer and base and any deck connecting protrusions of the composite bed, the protrusion reveals the top layer, and the maximum dimension of the protrusion is equal to or more than 4 mm of the top layer part and the height at which the protrusion reveals the top layer part is equal to or greater than 2 mm. Because the wallpaper has a protrusion, the user rotates under the box, leaning on the top left top right of the wallpaper.”

As can be seen, the system relates to wallpaper and does not mention a configuration that can provide a solution to the disadvantages mentioned above.

Abstract of the application numbered JP6712332B2, which emerged as a result of technical research is as follows; “The invention is a bionic prosthetic hand that includes: a piece of palm; a finger piece comprising an index finger component, a middle finger component, a ring finger component, a little finger component and a thumb component, wherein the index finger component, the middle finger component, the ring finger component and the little finger component are attached separately to the upper end of the palm piece in such a way that it can be opened separately.”

A hand used for prosthetic purposes is also disclosed in this application. Prosthetic hands are bionic materials designed only to help the person meet their daily activities and needs. It is not possible to use it for therapeutic touch purposes with this feature and it does not have the feature of giving information about the vital signs of the person

Abstract of the application numbered U.S. Ser. No. 10/219,919B2, which emerged as a result of technical research; “The invention provides a prosthetic hand with multiple grip types. The prosthetic finger units are operated insufficiently within and between the finger units using a differential mechanism arrangement. The locking action of the prosthetic thumb unit is coupled to the differential mechanism. While the user repositions the prosthetic thumb unit, the differential mechanism is affected, changing both the initial position and the force distribution of the prosthetic finger units. The present invention also provides an additive manufacturing molding method to do the same.”

As can be seen, said prosthesis has the ability to grip and is used for prosthesis purposes. For this reason, the prosthesis is a product that can be used by people who have lost their hands for any reason or who do not have a hand from birth. It is not possible to grasp the hand of unconscious individuals, to use the product in the evaluation of vital signs (body temperature, saturation value, pulse) of the same, and to give human warmth and support to the same.

As a result, due to the abovementioned disadvantages and the insufficiency of the current solutions regarding the subject matter, a development is required to be made in the relevant technical field.

### Aim of the Invention

The invention aims to provide a structure having different technical features which brings a new development in this field different from the embodiments used in the state of the art.

The main aim of the present invention is to reveal a therapeutic touching device that increases the moral support, makes the person more positive, accelerates the healing process and makes individuals feel happier in the health field, intensive care units, palliative care centers, nursing homes, operating rooms and all individuals living alone.

Another aim of the invention is to fulfill the need for fever meter, pulse oximeter and pulse meter equipment as well as the therapeutic touch need of the person.

In order to fulfill the above-described purposes, the present invention is a therapeutic touching device that increases the moral support, makes the person more positive, accelerates the healing process and makes individuals feel happier and shows the vital signs in the health field, intensive care units, palliative care centers, nursing homes, operating rooms and all individuals living alone, characterized in that, it comprises the following;
Pulse meter that is on the touching device and positioned to touch the patient's hand; enables the measurement of the patient's pulse, thermometer that allows the measurement of the patient's temperature, oximeter that allows the measurement of the oxygen level in the patient's blood,
   Microprocessor that provides data collection and evaluation via pulse meter, thermometer and oximeter,
   Checks whether said pulse, temperature and oxygen data can be collected,
   Provides a warning if said pulse, temperature and oxygen data are below or above a certain value,
   enables the touching device to be moved by means of motors,
Display panel controlled by a microprocessor, which provides the display of pulse, temperature and oxygen data and provides warning to the user.

The structural and characteristic features of the present invention will be understood clearly by the following drawings and the detailed description made with reference to these drawings and therefore the evaluation shall be made by taking these figures and the detailed description into consideration.

### FIGURES CLARIFYING THE INVENTION

FIG. 1 shows the angle states of the second bionic hand with the horizontal plane in the closed state, which is 60 degrees, and in the open state, which is 30 degrees.

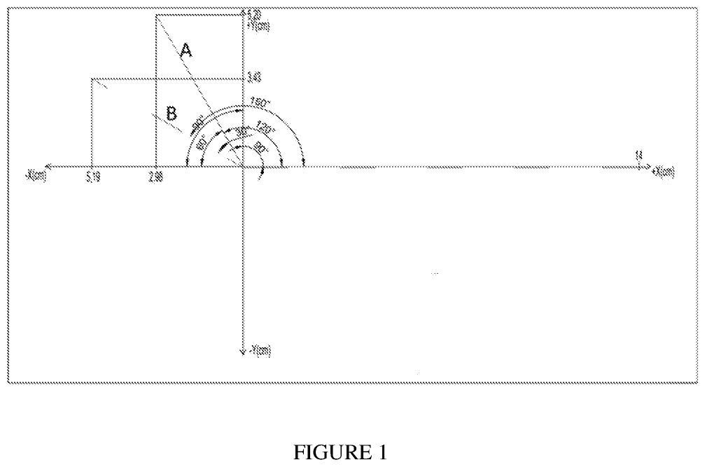

FIG. 2 shows the angle states of the second bionic hand with the horizontal plane in the closed state, which is 60 degrees, and in the open state, which is 30 degrees.

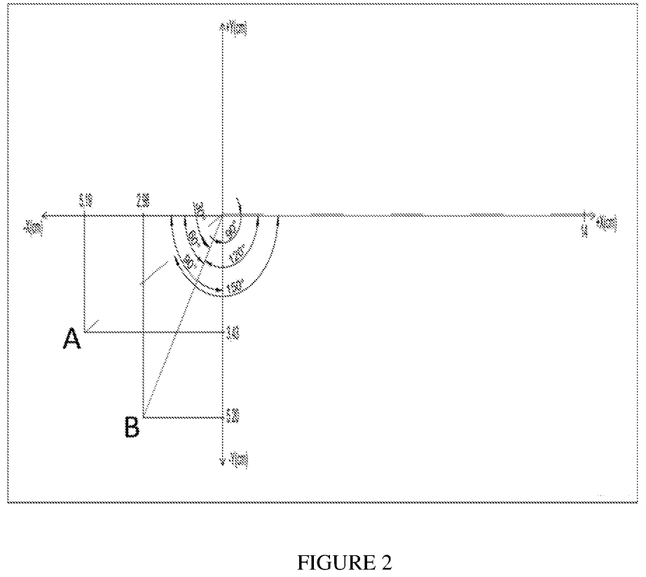

FIG. 3, is a detailed view of the inventive touching device.

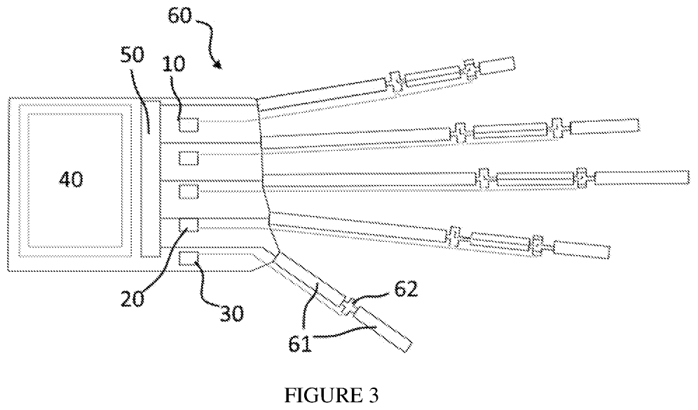

FIG. 4, is a side view of the inventive touching device.

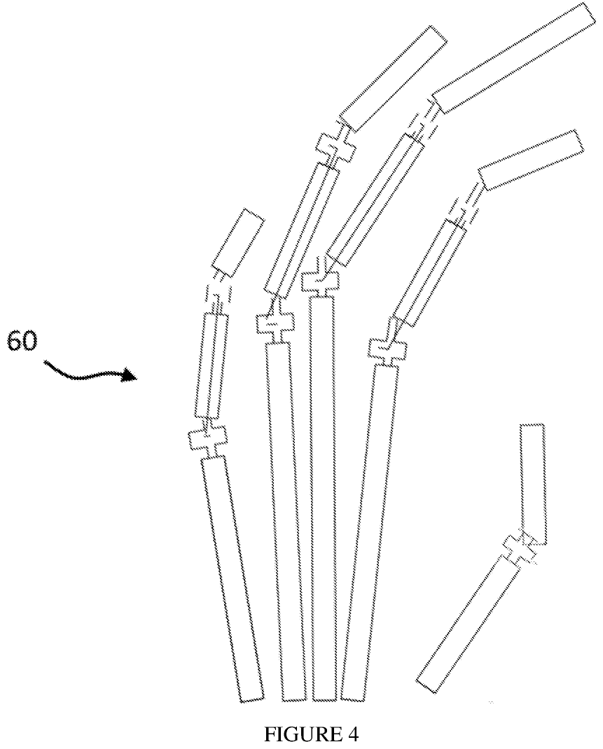

FIG. 5, is a side view of the inventive touching device from a different angle.

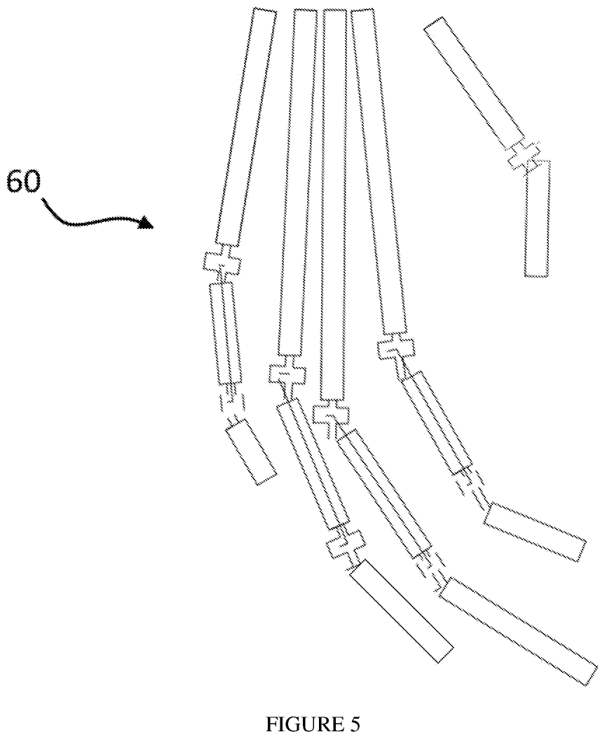

FIG. 6 is the illustration of the way of closing the inventive touching device with a human hand.

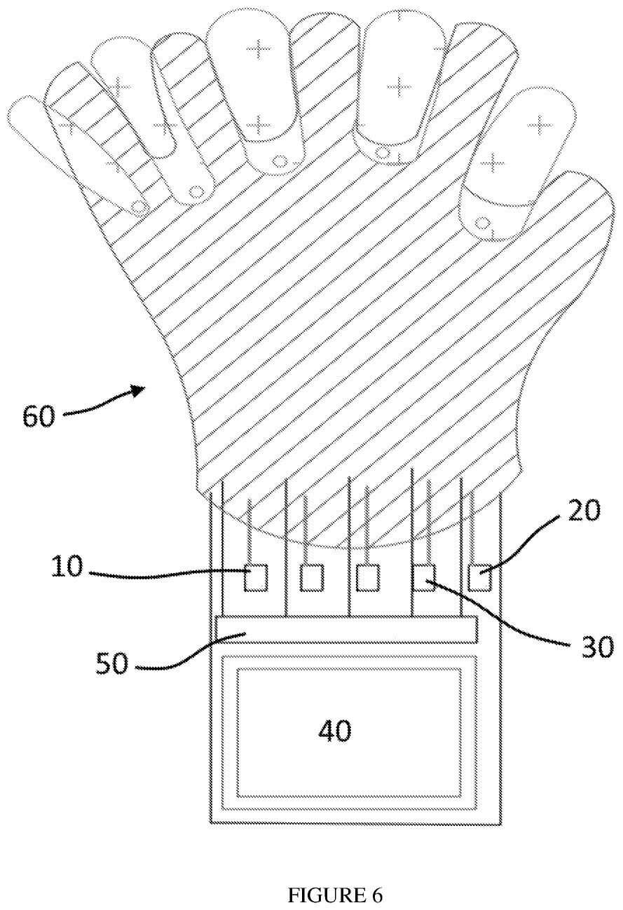

FIG. 7 is an illustrative view of the Microprocessor circuit.

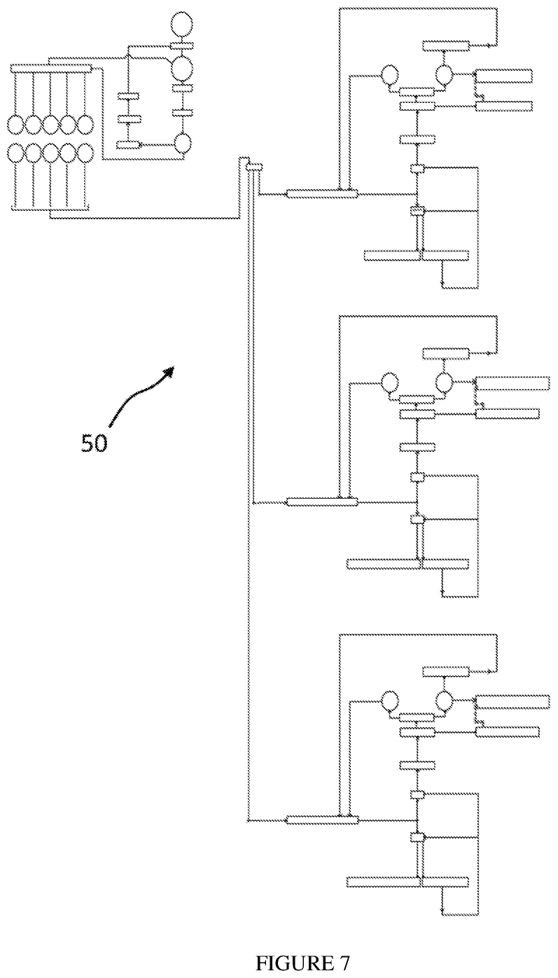

FIG. 8 is a view of the step motor structure.

FIG. 9 is a view of the operating principle of the step motor.

The figures are not required to be scaled and the details which are not necessary for understanding the present invention may be neglected. Moreover, the elements that are at least substantially identical or have at least substantially identical functions are shown by the same number.

### DESCRIPTION OF THE PART REFERENCES

10. Pulse meter
20. Thermometer
30. Oximeter
40. Display panel
50. Microprocessor
60. Touching device
61. Knuckles
62. Joints
70. Motor
R. Rotor
S. Stator
N. N pole
S. S pole
1. First end
2. Second end
3. Third end
4. Fourth end
5. Fifth end
6. Sixth end
SW1. First switch
SW2. Second switch
SW3. Third switch
SW4. Fourth switch
SW5. Fifth switch
A. The situation where the touching device stops holding the patient's hand
B. The situation where the touching device holds the patient's hand

### DETAILED DESCRIPTION OF THE INVENTION

In this detailed description, the preferred embodiments of the invention are described only for clarifying the subject matter in a manner such that no limiting effect is created.

The present invention relates to a therapeutic touching device (60) (bionic hand) that gives the warmth and support of a person, also shows his/her vital signs and can be used in the field of health, in the intensive care unit, in need of palliative care, in home care support, in elderly individuals living alone, in children and adults living alone, and in all individuals who need moral support with its abovementioned properties.

The inventive touching device (60) is contacted to the patient's hand in a way that it can grasp. There is a pulse meter (10), a thermometer (20) and an oximeter (30) on the touching device (60). When the touching device (60) grasps the patient's hand, the pulse meter (10), the thermometer (20) and the oximeter (30) are also positioned to touch the patient's hand. The pulse meter (10), thermometer (20) and oximeter (30) can preferably be positioned on the wrist or palm of the touch device (60).

Pulse meter (10) enables the measurement of the patient's pulse, thermometer (20) that allows the measurement of the patient's temperature, oximeter (30) that allows the measurement of the oxygen level in the patient's blood, The data collected by the pulse meter (10), the temperature meter (20) and the oximeter (30) are converted into digital signals and read by the microprocessor (50).

The microprocessor (50) first checks whether data is received from the pulse meter (10), thermometer (20) and oximeter (30) and whether their connections are correct. If the connections are negative, the ‘no’ cycle in the microprocessor (50) algorithm cycle is activated. If the connection is positive, the ‘Yes’ cycle starts. Microprocessor preferably uses PIC software.

These read values are processed in the microprocessor (50) as appropriate values between 60-100 minutes for pulse, appropriate values of 36-37.8 degrees for body temperature, and values above 95% for oxygen level. It determines those below and above these values as inappropriate values. The microprocessor (50) keeps the system running continuously with the help of the FLIPFLOP infinite conversion circuits that it contains in appropriate value ranges. In inappropriate value ranges, it performs the “no” cycle in the algorithm.

All data flow and outputs can be read from the display panel (40). The display panel (40) preferably consists of an LCD screen. In addition, a warning is given on the display panel (40) in said “no” cycle. The display panel (40) is controlled by the microprocessor (50).

The inventive touching device (60) can be moved electromechanically with two step motors. The control of the step motors is provided by the microprocessor (50). The touching device (60) continues in the ON/ON position for 30 seconds and OFF/OFF for 30 seconds until the system is stopped manually with the help of the “timer” software on the microprocessor (50). The ON position has a 60 degree angular position with the horizontal plane and the OFF position has an 30 degree angular position with the horizontal plane. It is the position in which the hand of a healthy or sick individual is placed between two bionic hands at an angular difference of 60 degrees with the horizontal plane. ON-OFF position times are fixed in software dimension. This complete system is provided by keeping the display panel (40) and the microprocessor (50) preferably connected to 220 V mains electricity. Apart from that, it can also be energized via the battery etc.

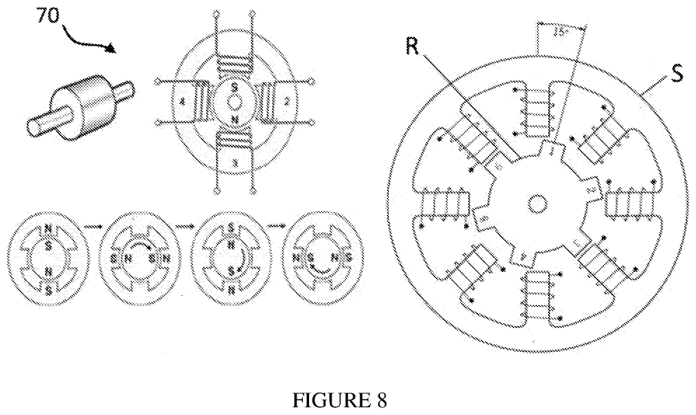

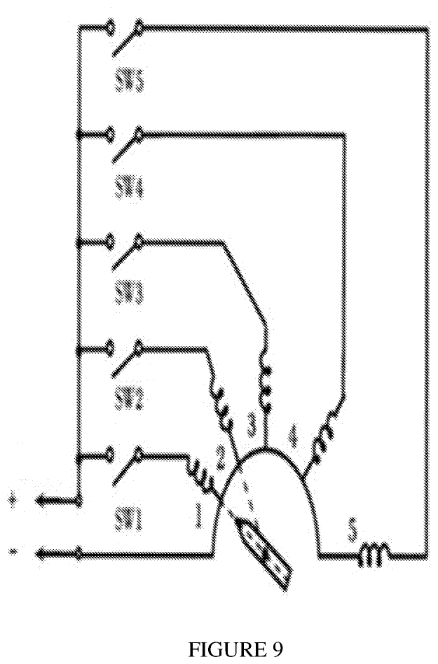

In FIG. 8, the structural shape of the step motor (70) is shown. All windings are positioned at 45 degree angles in 360 degree circular electromagnetic field (There are step motors with different angles.) In FIG. 9, the operating principle of the step motor (70) is explained. When electric current is applied to the second end (2), the 2-step angle motor will start. When electric current is applied to the fourth end (4), the 4-step angle motor will start. In this way, each angle level is dependent on the rotational speed of the motor and the trigger current. The aim of using the step motor (70) in the artificial human hand is to obtain the angular changes in the hand movements. In this way, when the motor is energized for 60 degree rotation, the motor will make a step rotation accordingly. The order of rotation of the step motors is provided by a microprocessor (50) based software electronic circuit.

The inventive touching device (60) is in the form of a bionic hand and is designed to give the patient a positive energy and raise his/her spirituality. Therefore, it consists of materials that will give the feeling of a real human hand. There are fingers on the touching device (60). The knuckles (61) on the fingers are connected to each other by finger joints (62). The equipment used in the touching device (60) are as follows; step motor (70), fishing line, human hand model mechanical parts, elastic material that will provide anatomical artificial human hand skin tissue to close the mechanical parts, the skin texture with the feeling of real human hand will be provided with ready-made artificial silicone elastic material. The temperature of the hand will be between 36-38 degrees, like the human body. Thus, the temperature of the hand will be measured with the thermometer (20) on the device, otherwise it will give a warning. Plates with high thermal insulation will be used so that excess heat in the device does not affect the external structure of the hand.

1. A therapeutic touching device that increases the moral support, makes the person more positive, accelerates the healing process and makes individuals feel happier and shows the vital signs in the health field, intensive care units, palliative care centers, nursing homes, operating rooms and all individuals living alone, the device comprising:
a pulse meter that is on the touching device and positioned to touch the patient's hand, a thermometer that allows the measurement of the patient's temperature, an oximeter that allows the measurement of the oxygen level in the patient's blood;
a microprocessor that provides data collection and evaluation via pulse meter, thermometer and oximeter;
wherein the microprocessor checks whether the said pulse, temperature and oxygen data can be collected,
wherein the microprocessor provides a warning if said pulse, temperature and oxygen data are below or above a certain value,
wherein the microprocessor enables the touching device to be moved by means of motors,
a display panel controlled by the microprocessor, which provides the display of pulse, temperature and oxygen data and provides warning to the user.

2. Touching device according to claim 1, wherein said motors are step motors.

3. Touching device according to claim 1, wherein said display panel comprises an LCD screen.

4. Touching device according to claim 1, wherein the pulse meter, thermometer and oximeter are positioned on the wrist or palm region.

5. Touching device according to claim 1, comprising two step motors that enable the touching device to be positioned at an angle of 30 and 60 degrees to the horizontal plane.

6. Touching device according to claim 1, wherein the microprocessor evaluates the appropriate value between 60-100 minutes for the heart rate, the appropriate value of 36-37.8 degrees for the body temperature, and the values above 95% for the oxygen level, and provides a warning from the display panel if these values are exceeded.

7. Touching device according to claim 1, the touching device comprising fingers consisting of knuckles connected by finger joints.

8. Touching device according to claim 1, wherein said touching device is a bionic hand grasping the patient's hand.
